(19) United States
(12) Patent Application Publication
(10) Pub. No.: US 2014/0158572 A1
(43) Pub. Date: Jun. 12, 2014

(54) Wound Care Dressing

(71) Applicant: Inventagon LLC, Nyack, NY (US)

(72) Inventor: Jarl Jensen, Nyack, NY (US)

(73) Assignee: Inventagon LLC, Nyack, NY (US)

(21) Appl. No.: 14/099,604

(22) Filed: Dec. 6, 2013

Related U.S. Application Data
(60) Provisional application No. 61/734,847, filed on Dec. 7, 2012.

(51) Int. Cl. A61F 15/00

(52) U.S. Cl. CPC A61F 15/002
USPC 206/441

(57) ABSTRACT

The present invention relates to a wound care dressing and package assembly. The assembly includes a first package substrate or layer to which a wound care dressing is attached. The wound care dressing includes a backing layer and a contact layer. A release structure is in contact with the contact layer on a side of the contact layer opposite the wound care dressing. The release structure is supported by a second package substrate or layer. A seal interconnects the first and second package layers, forming a sealed volume in which at least the wound care dressing and the release structure is contained. The wound care dressing advantageously releases from the backing layer at low forces, e.g., less than about 1N/linear inch.

### CROSS REFERENCE TO RELATED APPLICATIONS

This application claims priority under 35 U.S.C. §119(e) to U.S. Provisional Patent Application Ser. No. 61/734,847 filed Dec. 7, 2012, which is incorporated herein in its entirety by reference.

### FIELD

The present disclosure relates to a wound care dressing. More particularly, a wound care dressing that can be placed on a wound using a reduced number of steps as compared to conventional wound dressings is provided.

### BACKGROUND

Conventional wound care dressings require multiple steps to apply. For example, a wound care dressing is typically placed within a sterile package or envelope. In order to apply the dressing, the packaging is broken to allow the dressing to be accessed. The dressing is then removed from the packaging. Often, a release liner then needs to be removed, before the dressing is placed on the wound. In the case of a thin film wound dressing, a frame carrying the dressing needs to be peeled off after the dressing is placed on the wound. Accordingly, multiple steps are typically required in order to apply a conventional thin film wound dressing.

The production or manufacture of a conventional thin film wound dressing is also complex, and relatively expensive. For example, in order to form a typical thin film wound dressing, components of the dressing are formed using rotary die cutting techniques. The different components of the dressing must then be registered with one another, and assembled. In addition to requiring multiple precision forming and registration steps, conventional techniques can result in the creation of a relatively large amount of waste material.

### SUMMARY

In accordance with embodiments of the present disclosure, a wound care dressing that can be applied in a reduced number of steps, and that can be produced more economically than many conventional wound care dressings, is provided. More particularly, a wound care dressing assembly is provided that can include first and second package layers that are bonded to one another about a periphery of the first and second package layers by a seal, to form a sealed volume. A wound care dressing is located within the sealed volume. The wound care dressing can be interconnected to and/or formed on the first package layer. A release structure is formed on the second package layer. The release structure may be a continuous layer or may include multiple three-dimensional features or support elements that have the effect of reducing the surface area of the associated package layer. The wound care dressing can include a backing layer and a contact layer. The contact layer can cover all or a portion of the surface of the side of the backing layer facing the release structure. Moreover, the contact layer and/or a surface of the wound care dressing can be in contact with the release structure.

Methods for applying a wound care dressing according to the present disclosure include separating the first package layer from the second package layer. More particularly, the first package layer can be peeled away from the second package layer by breaking the seal between the first and second package layers. The wound care dressing and in particular at least most of the contact layer, which can comprise an adhesive, remains attached to the first package layer. The wound care dressing can then be applied by placing the adhesive side of the wound care dressing over the wound. The application of the wound care dressing can then be completed by removing the first package layer from the wound care dressing, leaving the wound care dressing in place over the wound. The first package layer and the second package layer, with an associated release structure, that was separated from the first package layer and the wound care dressing can then be discarded.

In accordance with still other embodiments of the present disclosure, a wound care dressing can be formed using a method that includes forming or placing a backing layer on a first package layer, and covering or substantially covering the dressing with a layer of adhesive or other contact material. The method also includes forming a release structure on a second package layer. The two package layers can then be interconnected to one another about a periphery of the package layers to form a sealed volume containing the backing layer, the adhesive or contact material, and the release structure.

Additional features and advantages of embodiments of the present disclosure will become more readily apparent from the following description, particularly when taken together with the accompanying drawings.

### BRIEF DESCRIPTION OF THE DRAWINGS

FIG. 1 depicts a wound care assembly with release structure peaks in accordance with embodiments of the present disclosure in plan view;

FIG. 2 depicts the wound care assembly of FIG. 1 in a cross-sectional view taken along section line A-A;

FIG. 3 depicts a wound care dressing in accordance with embodiments of the present disclosure in cross-section, after separation of the first and second package layers;

FIG. 4 depicts a wound care dressing in accordance with embodiments of the present disclosure in cross-section, applied to the skin of a patient;

FIG. 5 depicts a cross section of a wound care assembly with a uniform release structure;

FIG. 6 depicts a wound care assembly in accordance with embodiments of the present disclosure in cross-section, after separation of the first and second package substrates;

FIG. 7 depicts a wound care dressing in accordance with embodiments of the present disclosure in cross-section, applied to the skin of a patient;

FIG. 8 is a flowchart depicting aspects of a process for applying a wound care dressing in accordance with embodiments of the present disclosure; and

FIG. 9 is a flowchart depicting aspects of a process for forming a wound care dressing in accordance with embodiments of the present disclosure.

### DETAILED DESCRIPTION

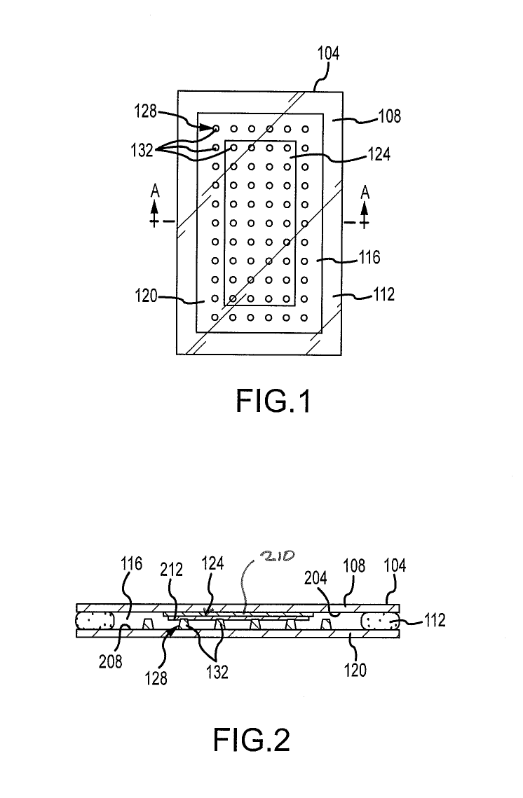

FIG. 1 depicts a wound care dressing assembly 104 in accordance with embodiments of the present disclosure in plan view. The wound care dressing assembly 104 includes first or top package layer 108. As shown in FIG. 1 for purposes of clarity, the first package layer 108 can be transparent, translucent, opaque, non-transparent, or the like. A seal 112 is formed about a perimeter of the assembly 104, creating a sealed volume 116 between the first package layer 108 and a second or bottom package layer 120. A wound care dressing 124 can be formed on and/or interconnected to a surface of the first package layer 108 that faces a surface of the second package layer 120, such that the wound care dressing 124 is located within the sealed volume 116. The wound care dressing 124 may be any shape or size. A side of the wound care dressing 124 facing the second package layer 120 can be in contact with a release or support structure 128 formed on and/or interconnected to the surface of the second package layer 120. The release structure 128 can also be located within the sealed volume 116. As will be described in greater detail elsewhere herein, the release structure 128 can comprise a structure that extends from a surface of the second package layer 120, to provide a reduced surface area in contact with the wound care dressing 124 and/or an adhesive associated with the wound care dressing 124.

FIG. 2 depicts a cross-section of the wound care dressing assembly 104 of FIG. 1, taken along section line A-A in FIG. 1. As seen in FIG. 2, the first package layer or substrate 108 generally extends across the entire width, and also the length, of the wound care dressing assembly 104. The seal 112 connects a first or inner surface 204 of the first package layer 108 to a first or inner surface 208 of the second package layer or substrate 120, such that a sealed volume 116 is defined between the first package layer 108, the second package layer 120, and the seal 112. In some embodiments, a liner layer (not shown) may be placed on the first or inner surface 204, 208 of the first package layer 108 and/or the second package layer 120. The liner layer may be a polymeric material, including but not limited to polyester, polyurethane, polypropylene or the like. The liner layer may cover all, or a portion of the first or inner surface 204, 208 of the first package layer 108 and/or the second package layer 120. The second package layer 120 generally has a width, and also a length, that is the same or similar to that of the first package layer 108. The wound care dressing 124 is connected to the first surface 204 of the first package layer 104. The wound care dressing 124 can include or be associated with a backing layer 210 and a contact layer 212. As an example, the backing layer 210 of the wound care dressing can comprise a side of the wound care dressing that is connected to the first surface 204 of the first package layer, while the contact layer 212 comprises some or all of a side of the wound care dressing 124 adjacent the first surface 208 of the second package layer 120. In addition, the contact layer 212 and/or the backing layer 210 of the wound care dressing 124 can be in contact with the release structure 128. Moreover, as shown in FIG. 2, the release structure 128 extends from the first surface 208 of the second package layer 120, to provide a support for the wound care dressing 124. In addition, the release structure 128 can present a reduced surface area to the surface of the wound care dressing 124 facing the release structure 129. More particularly, embodiments of the present invention provide a release structure 128 with support elements 132 that create a reduced surface area as compared to a configuration in which the wound care dressing area and/or the adhesive 212 is entirely in contact with the first surface 208 of the second package layer 120. As examples, but without limitation, the backing layer 210 may comprise a polyurethane material, and the contact layer 212 may comprise an adhesive, such as an acrylic adhesive.

As shown in FIGS. 1 and 2, the release structure 128 can comprise individual support elements 132. The individual support elements 132 can be projections that extend from the first surface 208 of the second package layer 120. Moreover, the release structure 128 can include support elements 132 that are interconnected to one another. For instance, the release structure 128 can comprise a unitary structure. Alternatively, some or all of the support elements 132 can be separate from other support elements 132. In accordance with further embodiments, the support elements 132 extend vertically from the first surface 208 of the second package layer, or from a portion of the release structure 128 interconnecting support elements 132, for a distance that is greater than the thickness of the contact layer 212. The support elements can be provided in various shapes, including but not limited to cones, cylinders, rectangles, squares, spheres, hemispheres, truncated cones, ridges, or triangles. In accordance with still other embodiments, the release structure 128 can comprise ridges, corrugations or other projections that create a three-dimensional surface to partially contact the contact layer 212.

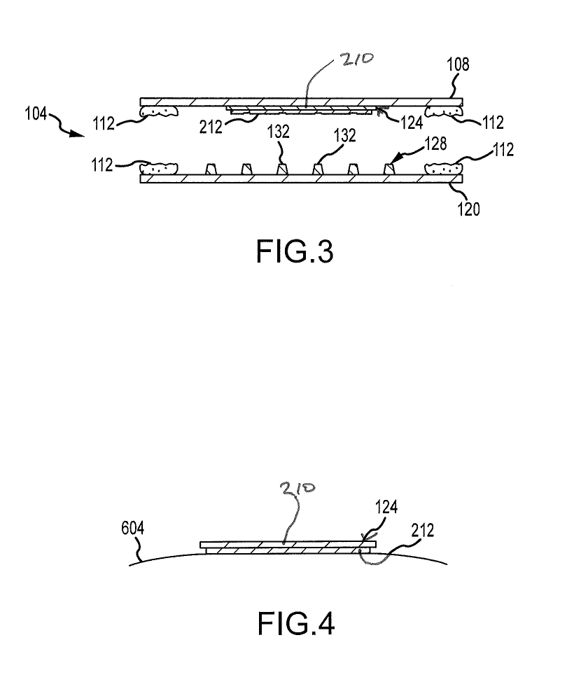

FIG. 3 depicts a wound care dressing assembly 104 with the first package layer 108 separated from the second package layer 120. In general, the wound care dressing 124 remains interconnected to the first package layer 104. The release structure 128 remains interconnected to the second package layer 120. As can be appreciated by one of skill in the art after consideration of the present disclosure, by separating the first package layer 104 from the second package layer 120, for example by breaking the seal 112 formed about the peripheries of the first package layer 104 and the second package layer 120, the sealed volume 116 no longer exists. Moreover, the contact layer 212, comprising some or all of the surface of the wound care dressing 124 opposite the first package layer 108, is exposed.

FIG. 4 depicts the wound care dressing 124 in cross-section, attached to the skin 604 of a patient by the contact layer 212. The first package layer 108 has been removed.

The shape and size of the wound care dressing assembly 104 and/or the wound care dressing 124 may be any suitable shape or size, including a square, rectangle, circle, oval, triangle, strip or any suitable shape in any desired size.

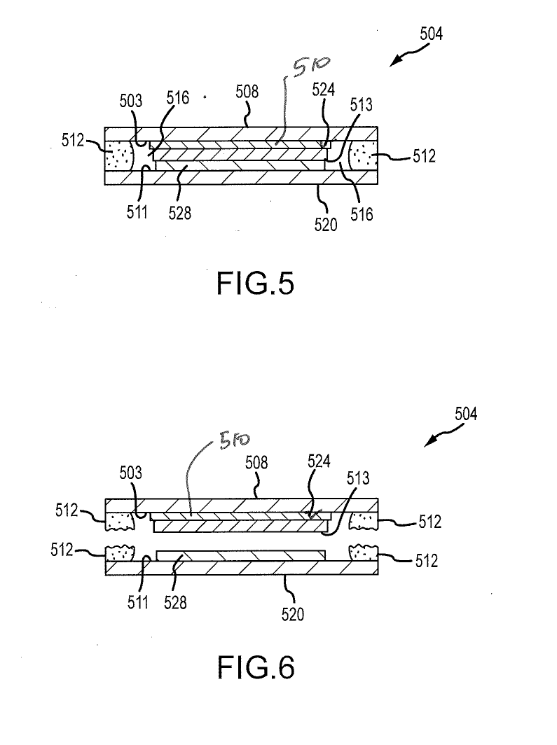

FIG. 5 depicts a cross-section of the wound care dressing assembly 504. As seen in FIG. 5, the first package substrate 508 generally extends across the entire width, and also the length, of the wound care dressing assembly 504. The seal 512 connects a first or inner surface 503 of the first package substrate 508 to a first or inner surface 511 of the second package substrate 520, such that a sealed volume 516 is defined between the first package substrate 508, the second package substrate 520, and the seal 512. In some embodiments, a liner layer (not shown) may be interconnected to the first or inner surface 503, 511 of the first package substrate 508 and/or the second package substrate 520. The liner layer may be a polymeric material, including but not limited to polyester, polypropylene or the like. The liner layer may cover all, or a portion of the first or inner surface 503, 511 of the first package substrate 508 and/or the second package substrate 520. The second package substrate 520 generally has a width, and also a length, that is the same or similar to that of the first package substrate 508. The wound care dressing 524 is connected to the first surface 504 of the first package substrate 504. The wound care dressing 524 can include or be associated with a backing layer 510 and a contact layer 513. The contact layer 513 can comprise some or all of a side of the wound care dressing 524 adjacent the first surface 511 of the second package substrate 520. The wound care dressing 524 may be any shape or size. In some instances, the contact layer 513 covers the entire surface of the backing layer 510, or is slightly smaller than the backing layer 510. In addition, the contact layer 513 and/or the backing layer 510 can be in contact with the release structure 528 as shown in FIG. 5. Moreover, as shown in FIG. 5, the release structure 528 extends from the first surface 511 of the second package substrate 520, to provide a support for the wound care dressing 524, for instance through the contact layer 513, and has a reduced surface friction. The release structure 528 may be slightly larger, slightly smaller, or the same size as the wound care dressing 524 and/or the contact layer 513.

FIG. 6 depicts a wound care dressing assembly 504 with the first package substrate 508 separated from the second package substrate 520. In general, the wound care dressing 524 remains interconnected to the first package substrate 508. The release structure 528 remains interconnected to the second package substrate 520. As can be appreciated by one of skill in the art after consideration of the present disclosure, by separating the first package substrate 508 from the second package substrate 520, for example by breaking the seal 512 formed about the peripheries of the first package substrate 508 and the second package substrate 520, the sealed volume 516 no longer exists. Moreover, the contact layer 513 forming some or all of one surface of the wound care dressing 524 is exposed.

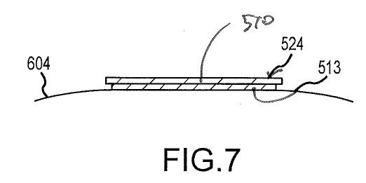

FIG. 7 depicts the wound care dressing 524 in cross-section, attached to the skin 504 of a patient by the contact layer 513. The first package substrate 508 has been removed.

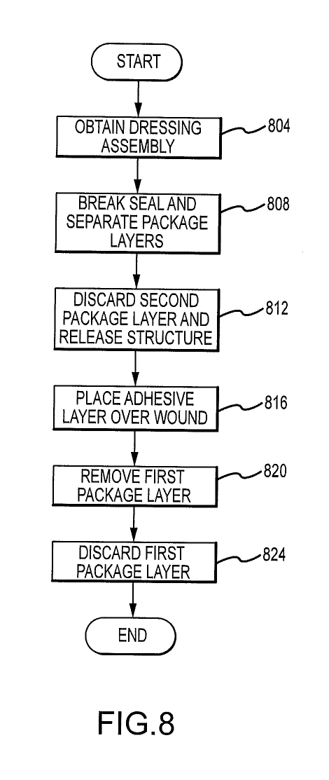

FIG. 8 depicts aspects of a process for applying a wound care dressing 124, 524 to the skin 604, 504 of a patient in accordance with embodiments of the present disclosure. Initially, at step 804, a wound care dressing assembly 104, 504, such as depicted in FIGS. 1 and 2 and FIGS. 5 and 6, respectively, is obtained. At step 808, the seal 112, 512 is broken, and the first package layer or substrate 108, 508 is separated from the second package layer or substrate 120, 520. In accordance with embodiments of the present disclosure, the backing layer 210, 510, and the contact layer 212, 513 comprising the wound care dressing 124, 524 remain with the first package layer or substrate 104, 508. The backing layer 210, 510 and the associated contact layer 212, 513 are released from the second package layer or substrate 120, 520 and the release structure 128, 528 when the first package layer or substrate 108, 508 is disconnected from the second package layer 120, 520. More particularly, the bond between the backing layer 210, 510 and the first package or substrate 108, 508 is greater than the bond between the contact layer 212, 513 and the release structure 128, 528 that is connected to the second package layer or substrate 120, 520. In some embodiments, the reduced surface area of the release structure 128 in contact with the contact layer 212 is reduced compared to the surface area of the backing layer 210, 510 in contact with the contact layer 212. In other embodiments, materials of the release layer 128, 528 are chosen such that the bond between the contact layer 212, 513 and the release layer 128, 528 is weak. The second package layer or substrate 120, 520 and the interconnected release structure 128, 528 can then be discarded (step 812).

At step 816, the contact layer 212, 513 is placed over a wound on the patient's skin 604, 504. As can be appreciated by one of skill in the art after consideration of the present disclosure, the wound care dressing 124, 524 can be applied to the skin 604, 504 of a patient by handling the first package layer or substrate 104, 508, and without requiring contact with the wound care dressing 124, 524 by a medical practitioner or other person applying the wound care dressing 124, 524. At step 820, the first package layer 104, 508 can be removed from the wound care dressing 124, 524. As a result, the wound care dressing 124, 524 and the contact layer 212, 513 remain attached to the patient's skin 604, 504. The first package layer 104, 508 can then be discarded (step 824).

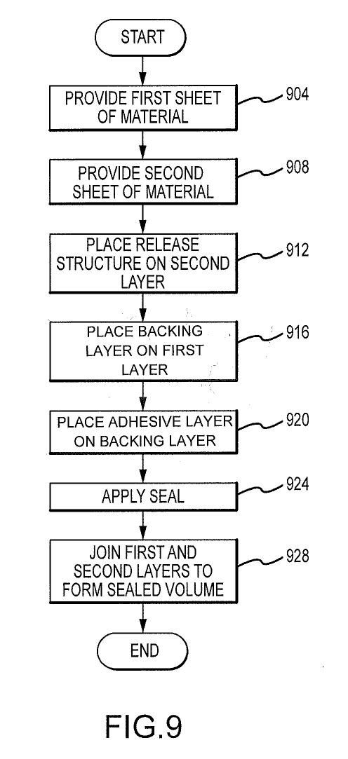

With reference now to FIG. 9, a flowchart depicting aspects of a process for forming a wound care dressing assembly 104, 504 in accordance with embodiments of the present disclosure is illustrated. Initially, at step 904, a first sheet of material is provided. The first sheet of material will be used to form the first package layer or substrate. In accordance with embodiments of the present disclosure, the first package layer or substrate can include, but is not limited to, a polymeric film, such as polyethylene, polyester, polyurethane, or polypropylene, paper, coated paper, or the like. At step 908, a second sheet of material is provided. The second sheet of material will be used to form the second package layer or substrate 120, 520. The second package layer or substrate 120, 520 can be formed from the same or different material or materials used to form the first package layer 104, 508. At step 912, the release structure 128, 528 is interconnected to and/or formed on the first surface 208, 511 of the second package layer or substrate 120, 520. In accordance with embodiments of the present disclosure, the release structure 128, 528 is a release material such as, for example only, silicone, fluorine, or polymeric fluorine material. The release structure 128, 528 extends from the first surface 208, 511 of the second package layer or substrate 120, 520. The release structure 128, 528 can be formed using various methods, including printing processes. In some embodiments, a plurality of support elements 132 comprising the release structure 128 are formed on the first surface 208 of the second package layer 120. In other embodiments, the release structure 528 is a continuous part, and may have a substantially uniform thickness across a portion of the second package substrate 520.

At step 916, the backing layer 210, 510 of the wound care dressing 124, 524 is formed on the first surface 204, 503 of the first package layer or substrate 108, 508. The backing layer 210, 510 can include, but is not limited to, a polymeric material, such as a polyurethane film. Moreover, the backing layer 210, 510 can be cast onto the first surface 204, 503 of the first package layer or substrate 208, 508. At step 920, a contact layer 212, 513 is applied to the backing layer 210, 510 of the wound care dressing 124, 524. In some embodiments, the contact layer 212, 513 may comprise an adhesive, a polyurethane material, a hydrocolloid material, nicotine, antimicrobial, antibacterial, silver, antibiotic, pharmaceutical, including but not limited to birth control, combinations thereof or the like. The backing layer 210, 510 and/or the contact layer 212 can incorporate or hold an absorbent foam, hydrogel, or other material. In accordance with further embodiments, a release coating can be applied to portions of the first package layer or substrate 108, 508 that are not occupied by the wound dressing 124, 524 and/or the contact material 212, 513, and/or to the surface of the second package layer or substrate 120, 520. At step 924, a seal 112 is applied to the first surface 204, 503 of the first package layer or substrate 108, 508, and/or the first surface 208, 511 of the second package layer or substrate 120, 520, about the perimeter of the first surface 204, 503 and/or 208, 511 of the first and/or second package layers or substrate 104, 508 and 120, 580, respectively. The first package layer or substrate 104, 508 is then joined to the second package layer or substrate 120, 520 by the seal 112, 512, to form the sealed volume 116, 516 containing the wound care dressing 124, 524, including the backing layer 210, 510 and the contact layer 212, 513, and the release structure 128, 528 (step 928). The seal 112, 512 can comprise an applied material, such as an acrylic, a pressure sensitive adhesive, an adhesive, a co-adhesive, glue, or a weld between the first package layer or substrate 108, 508 and second package layer or substrate 120, 520. The process for forming a wound care dressing assembly 104, 504 is then complete.

One advantage of the present invention is the ability to put multiple zones of materials, such as adhesives, in the wound care assembly. The zones may comprise different materials, adhesives or/and different adhesive levels. This ability is advantageous because different adhesive strengths are advantageous at different locations within the wound care assembly. Near the edge of the wound care assembly, for example at the seals, a high adhesive strength material is advantageous so the perimeter is sufficiently sealed. Near the interior of the wound care assembly, a lower strength adhesive or contact material is advantageous so that the wound care dressing may be removed from the wound care assembly prior to application to the patient and/or from the patient after use so as to decrease pain when the wound care dressing is removed from the patient, while having sufficient strength to keep the wound dressing in contact with the patient during use. The present invention allows multiple zones of adhesive to be applied without additional processing, such as exposing a portion of the wound care assembly to ultraviolet (UV) light or other process, to change the properties of the adhesive for the exposed portion (for example to decrease the adhesive strength in some areas of the wound care assembly).

Furthermore, different types of materials may be applied throughout the wound care assembly. Thus, by way of example, one zone of the wound care assembly may have an adhesive surface for drug delivery, i.e. the contact layer, and another zone exterior to the contact layer that does not have a drug delivery adhesive, i.e. the seal. The seal and the contact layer may have different characteristics or performance features. In another example, the contact layer and/or the dressing may have a high absorption capability for wound fluid, while the seal material of the assembly has a low absorption capability.

In some embodiments, the present invention is made with the use of a three-dimensional printer. An advantage of this manufacturing method is that the layers may be built on the substrate such that adhesive layers are not required between each layer to hold the each layer in place. Thus, for example, no adhesive is required between the first package substrate and the wound care dressing.

Another advantage of the present invention is that the first package layer with or without the liner, from the wound dressing with minimal force, in many cases less than about 1 N/linear inch. In some embodiments, the force to remove the wound dressing from the first package layer or substrate is less than about 10 N. In some embodiments, the force to remove the wound dressing from the first package layer was less than about 10 N, 9 N, 8 N, 7 N, 6 N, 5 N, 4 N, 3 N or about 1 N.

### Examples

A test was performed to determine the force required to remove a wound dressing from a test panel. The wound dressing was removed at an approximately 90° angle to a standard steel panel. Test specimens were cut to approximately 25 mm in width and 100 mm in length or the length of the product. The specimens were conditioned at room temperature for approximately 8 hours. The panel was cleaned prior to testing and allowed to dry for at least 10 minutes. At one end of the specimen, approximately 20-30 mm of the adhesive which will be affixed to the clamp were separated. The remainder of the specimen is removed from its liner and placed above the test panel. The entire specimen is allowed to fall gently onto the panel. Then a weighted roller is placed on the specimen and rolled once in each direction over the specimen. Care was taken to minimize air bubbles between the sample and the test specimen and not to apply additional force on the roller during the passes over the specimen. Approximately 15 mm of the specimen is attached to the tensile tester and the specimen is pulled from the panel at an angle of approximately 90°.

Table summarizes the results of the peel test. The values, in Newtons, were taken over time as the dressing was removed in a linear motion. The peak force occurred as the initial point that the dressing lifted from the package layer or substrate.

| | Test/Force | | | | | | | | |
|---|---|---|---|---|---|---|---|---|---|
| | 1 | 2 | 3 | 4 | 5 | 6 | 7 | 8 | 9 |
| | 0 | −0.21 | −0.33 | 0 | −0.26 | −2.54 | −6.3 | −2.97 | −2.02 |
| | −1.24 | −0.37 | −0.52 | −0.26 | −0.33 | −9.25 | −6.61 | −3.57 | −3.95 |
| | −0.54 | −0.35 | −0.49 | 0.08 | −0.36 | −9.38 | −1.27 | −0.17 | −1.68 |
| | −6.4 | −0.92 | −0.95 | −4.82 | −0.73 | −0.38 | −1.18 | −0.1 | −1.6 |
| | −6.44 | −0.93 | −0.89 | −3.69 | −0.5 | −0.38 | −0.71 | −0.2 | −1.14 |
| | −1.37 | −6.39 | −4.26 | −0.26 | −2.32 | −0.42 | −0.82 | −0.23 | −1.14 |
| | −0.99 | −4.78 | −2.72 | −0.35 | −2.09 | −0.42 | −0.54 | −0.14 | −0.98 |
| | −0.37 | −0.41 | −0.77 | −0.43 | −4.68 | −0.43 | −0.54 | −0.17 | −1.1 |
| | −0.38 | −0.44 | −0.76 | −0.42 | −3.74 | −0.47 | −0.54 | −0.22 | −0.82 |
| | −0.33 | −0.46 | −0.78 | −0.32 | −0.28 | −0.43 | −0.52 | −0.22 | −0.76 |
| | −0.31 | −0.47 | −0.79 | −0.34 | −0.29 | −0.43 | −0.45 | −0.28 | −0.57 |
| | −0.29 | −0.56 | −0.79 | −0.13 | −0.37 | −0.43 | −0.55 | −0.27 | −0.54 |
| | −0.33 | −0.61 | −0.78 | −0.13 | −0.38 | −0.42 | −0.07 | −0.29 | −0.7 |
| | −0.26 | −0.63 | −0.75 | −0.02 | −0.47 | −0.45 | −0.2 | −0.27 | −0.65 |
| | −0.32 | −0.61 | −0.82 | −0.1 | −0.42 | −0.46 | −0.01 | −0.26 | −0.55 |
| Peak | 6.44 | 6.39 | 4.26 | 4.82 | 4.68 | 9.38 | 6.61 | 3.57 | 3.95 |

The peak force to remove the dressing from the first package layer or substrate was about 9.38 N.

Although processes described herein have been presented with a particular ordering of steps, different sequences and/or collections of steps are possible.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A wound care dressing assembly, comprising:
a first package substrate;
a wound care dressing, including:
a backing layer, wherein a first surface of the backing layer is interconnected to a first surface of the first package substrate; and
a first contact layer, wherein the first contact layer is placed on or adjacent
a second surface of the backing layer;
a release structure, wherein the first contact layer of the wound care dressing is in contact with the release structure;
a second package layer, wherein a first surface of the second package layer is interconnected to the release structure, and wherein the first surface of the first package layer faces the first surface of the second package layer; and
a seal, wherein the first package layer is interconnected to the second package layer by the seal, wherein the wound care dressing is contained within a sealed volume defined by the first and second package layers and by the seal interconnecting the first and second package layers.

2. The assembly of claim 1, wherein the release structure is a three dimensional structure.

3. The assembly of claim 1, wherein the release structure comprises a release material selected from the group consisting of a silicone, a fluorine, or a polymeric fluorine.

4. The assembly of claim 2, wherein the release structure slightly larger than the wound care dressing.

5. The assembly of claim 1, wherein the release structure is has a uniform thickness.

6. The assembly of claim 1, wherein the contact layer comprises an adhesive.

7. The assembly of claim 1, wherein the seal comprises a material that is different from a material of the contact layer, and wherein the material of the seal is not treated with a UV process to change properties of the seal.

8. The assembly of claim 1, wherein the contact layer comprises a material selected from the group consisting of an adhesive, a polyurethane, a hydrocolloid, nicotine, an antimicrobial, an antibacterial, silver, an antibiotic, a pharmaceutical, and combinations thereof.

9. The assembly of claim 1, wherein the contact layer and the wound dressing layer have a high absorption capability.

10. The assembly of claim 1, wherein the first package layer and the second package layer are polypropylene.

11. The assembly of claim 1, wherein a force to remove the first package layer from the wound care dressing is less than about 10 N.

12. A wound care dressing assembly, comprising:
a first package substrate;
a wound care dressing, wherein a first surface of the wound care dressing is interconnected to a first surface of the first package substrate;
a release structure, wherein the wound care dressing is in contact with the release structure;
a second package substrate, wherein a first surface of the second package layer is interconnected to the release structure, and wherein the first surface of the first package substrate faces the first surface of the second package substrate; and
a seal, wherein the first package substrate is interconnected to the second package substrate by the seal, wherein the wound care dressing is contained within a sealed volume defined by the first and second package substrates and by the seal interconnecting the first and second package substrates.

13. The assembly of claim 12, wherein the wound care dressing includes a backing layer, and wherein the backing layer is polyurethane.

14. The assembly of claim 13, wherein the wound care dressing further includes a contact layer, wherein the contact layer is placed on or adjacent a second surface of the backing layer, and wherein the contact layer is an adhesive.

15. The assembly of claim 13, wherein the first package substrate is interconnected to the backing layer such that no adhesive is present between the first package substrate and the wound care dressing.

16. The assembly of claim 15, wherein the seal comprises a material that is different from a material of the contact layer, and wherein the material of the seal is not treated with a UV process to change properties of the seal.

17. The assembly of claim 15, wherein the contact layer comprises a material selected from the group consisting of an adhesive, a polyurethane, a hydrocolloid, nicotine, an antimicrobial, an antibacterial, silver, an antibiotic, a pharmaceutical, and combinations thereof.

18. The assembly of claim 12, wherein the release structure is a continuous piece of uniform thickness, and wherein the release structure is slightly larger in each dimension to the wound care dressing.

19. The assembly of claim 12, wherein a force to remove the first package substrate from the wound care dressing is less than about 10 N.

20. A wound care dressing assembly, comprising:
a first package substrate;
a wound care dressing, including:
a backing layer, wherein a first surface of the backing layer is interconnected to a first surface of the first package substrate; and
a contact layer, wherein the contact layer is placed on or adjacent a second surface of the backing layer;
a release structure, wherein the contact layer is in contact with the release structure;
a second package substrate, wherein a first surface of the second package layer is interconnected to the release structure, and wherein the first surface of the first package substrate faces the first surface of the second package substrate; and
a seal, wherein the first package substrate is interconnected to the second package substrate by the seal, wherein the wound care dressing is contained within a sealed volume defined by the first and second package substrates and by the seal interconnecting the first and second package substrates, and wherein a material for the seal and a material for the contact layer are different, and wherein neither an adhesive strength of the material for the seal nor the material for the contact layer are changed with a UV process.
